(19) United States
(12) Patent Application Publication
(10) Pub. No.: US 2004/0247568 A1
(43) Pub. Date: Dec. 9, 2004

(54) Method for formulation of microbial feed additives with feed

(76) Inventors: Frank Guerino, Monroe Township, NJ (US); Steven Holmes, Tinley Park, IL (US); James Skinner, Sevierville, TN (US); David Yates, Amarillo, TX (US)

Correspondence Address: CANTOR COLBURN, LLP, 55 GRIFFIN ROAD SOUTH, BLOOMFIELD, CT, 06002

(21) Appl. No.: 10/832,860

(22) Filed: Apr. 27, 2004

Related U.S. Application Data
(60) Provisional application No. 60/465,942, filed on Apr. 28, 2003.

(51) Int. Cl. A61K 45/00; A23K 1/165

(52) U.S. Cl. 424/93.4; 424/442

(57) ABSTRACT

Methods for the formulation of microbial feed additives with animal feed and methods of administering microbial feed additives are disclosed.

### CROSS-REFERENCE TO RELATED APPLICATIONS

This application claims the benefit of U.S. Provisional Application No. 60/465,942 filed Apr. 28, 2003.

### BACKGROUND

The present method is directed to methods for the formulation of microbial feed additives with animal feed, the animal feeds formed thereby, and methods of administering microbial feed additives.

Feed additives may be formulated with animal feed to provide additional nutrients, minerals, vitamins, medicaments, and other supplements. Such additive supplements are usually administered to each animal on a regular basis, in carefully controlled dosages, to ensure optimal benefit. Individual dosages are typically small due to the high potency of microingredients. Direct-fed microbials (DFM) are live microorganisms and/or their metabolites that may be added to livestock feed, for example, to provide beneficial microbes to livestock to help maintain normal gut flora, especially after stressful situations such as transport to a feedlot.

Feed additives may be added to either feed or watering systems accessible to the animals. A number of methods and apparatuses have been devised for accurately dispensing separately stored feed additives into a volume of carrier material, such as water, for dilution, dispersion, and suspension, and for transporting the resulting slurry into drinking water or feed rations shortly before the time of intended consumption. Methods for effective formulation of feed additives with feed desirably provide suitable dosage amounts, uniform delivery to feed sources, and allows even dispersal of additive in the feed, while at the same time minimizing waste, cost, and cleanup time. The stability and physical properties of DFM present special challenges to their formulation with animal feeds. Bacteria are sensitive to environmental conditions, for example oxygen (if anaerobic), moisture, pH, temperature extremes, and many chemicals. There accordingly remains a need in the art for a cost-effective, convenient method of delivery of DFM in a defined dose to animal feed, particularly livestock feed.

### SUMMARY

A method of formulating direct-fed microbials with animal feed comprises dispersing a direct-fed microbial composition into a solid or liquid carrier; and applying the dispersed direct-fed microbial composition to animal feed.

In another embodiment, a supplemented animal feed comprises animal feed and a direct-fed microbial composition formulated as described above.

In still another embodiment, a method for administering a direct-fed microbial composition to a population of animals comprises presenting the above-described supplemented animal feed to the animals for consumption.

### DRAWINGS

Referring now to the figure, which is an exemplary embodiment, and wherein the like elements are numbered alike:

FIG. 1 is a schematic of a generalized apparatus for dispersing DFM in a liquid carrier.

### DETAILED DESCRIPTION

The present method and compositions provide a convenient, cost effective means of dispersing a desired dose of DFM feed additives to animal feed. DFM compositions are often provided as lyophilized solids in sealed packages to enhance long-term stability, and may thus be mixed with feed in a dry state. It has been found by the inventors hereof that dispersal of the DFM composition in a dry or liquid carrier prior to applying to a feed is less sensitive to product concentrations, allows more uniform incorporation into the feed, and thus more uniform dosage, and promotes bacterial stability. The method is also more efficient, in that only the desired amounts of DFM are delivered, and equipment clean up is easier. Dispersion and delivery by a liquid dispersal apparatus is particularly preferred, as it provides improved results even over dry DFM compositions that have been formulated to enhance dry delivery. However, in the absence of such equipment, DFM compositions that have been formulated to enhance dry delivery produce acceptable results that are improved over DFM compositions that have not been formulated.

The terms “a” and “an” herein do not denote a limitation of quantity, but rather denote the presence of at least one of the referenced items. All ranges disclosed herein are inclusive and combinable.

DFM compositions for use as feed additives include a microbial culture, optionally together with the metabolites (e.g., proteins, including enzymes, vitamins, carbohydrates, amino acids, and the like) produced by the culture. As mentioned, DFM compositions can be used to provide beneficial microbes to livestock or other animals to help maintain normal gut flora, for example. DFM compositions can also be used to replace undesirable, e.g., pathogenic bacteria. Suitable DFM for use as a feed additive include, for example, Aspergillus niger, Aspergillus oryzae, Bacillus coagulans, Bacillus lentus, Bacillus licheniformis (ALCARE available from Alpharma Inc., NCTC 13123), Bacillus pumilus, Bacillus subtilis, Bacteroides amylophilus, Bacteroides capillosus, Bacteroides ruminocola, Bacteroides suis, Bifidobacterium adolescentis, Bifidobacterium animalis, Bifidobacterium bifidum, Bifidobacterium infantis, Bifidobacterium longum, Bifidobacterium thermophilum, Enterococcus faecium, Enterococcus intermedius, Enterococcus cremoris, Enterococcus diacetylactis, Enterococcus lactic, Enterococcus thermonphilus, Escherichia coli, (“E. coli”), Lactobacillus acidolphilus, Lactobacillus brevis, Lactobacillus buchneri (cattle only), Lactobacillus bulgaricus, Lactobacillus casei, Lactobacillus cellobiosus, Lactobacillus curvatus, Lactobacillus delbruekii, Lactobacillus fermentum, Lactobacillus helveticus, Lactobacillus lactis, Lactobacillus plantarum, Lactobacillus reuteri, Leuconostoc mesenteroides, Pediococcus acidilacticii, Pediococcus cerevisiae (damnosus), Pediococcus pentosaceus, Propionibacterium freudenreichii, Propionibacterium shermanii, Saccharomyces cerevisiae, and the like.

A preferred DFM composition includes certain bacterial strains of E. coli that help provide beneficial bacteria while at the same time excluding pathogens such as diarrheagenic E. coli strains and Salmonella DT104 from the animal's gut. Such strains of beneficial bacteria may be described as “competitive exclusion organisms” that aid in reducing or eliminating disease-producing pathogens that may be present in the animal. Exemplary beneficial strains of E. Coli DFM compositions are described in U.S. Pat. No. 5,965,128 to Doyle et al., which is incorporated by reference herein in its entirety. The preferred strains include E. coli 271, ATCC accession number 202020; E. coli 786, ATCC accession number 202018; and E. coli 797, ATCC accession number 202019, useful alone or in combination.

The amount of DFM incorporated into the feed will vary depending on the composition, animal, desired dosage, purpose, and like considerations, and is readily determined by one of ordinary skill in the art.

The DFM composition may optionally include or be used in combination with one or more additional feed additives, for example organic acids, phospholipids, enzymes, vitamins, carotenoids, prebiotics, immunostimulants, herbs/botanicals, ionophores, anti-microbials, or a mixture comprising at least one of the foregoing types of additional feed additives. Prebiotics are nondigestible feed ingredients that may selectively stimulate the growth and/or activity of one or a limited number of beneficial bacterial species present in the animal's gut. Exemplary prebiotics include nondigestible oligosaccharides including inulins and their derivatives such as fructo-oligosaccharides. Suitable organic acids include, for example, amino acids, propionic acid, formic acid, fumaric acid, citric acid, acetic acid, lactic acid, and the like. Exemplary herbs/botanicals include garlic, Echinacea, peppermint, oregano, radish, and the like. Suitable ionophores include monensin, lasalocid, laidlomycin, and the like. Anti-microbials include tylosin, chlortetracycline, oxytetracycline, and the like. Additional additives may be incorporated into the feed at any time in the feed preparation, for example, before, with, or after incorporation of the DFM composition.

In the practice of the present method, a DFM composition is first dispersed in a dry or liquid carrier to form a dispersed DFM composition. As used herein “dispersal” includes the formation of solid mixtures and liquid dispersions, solutions, suspensions, emulsions, and the like, to achieve a readily flowable state. Preferably, a first measured amount of the DFM composition is dispersed into a second measured amount of the carrier, such that the amounts of each are known. As used herein, “known amount(s)” is defined as measured amounts as measured by gravimetric or volumetric measuring equipment.

A number of suitable dry carriers having advantageous flow properties for dispersal of the DFM composition are available, and may be organic or inorganic. Suitable inorganic carriers that do not adversely affect the stability of the DFM compositions include, for example, salts such as calcium carbonate, calcium sulfate, and the like. Suitable organic carries include lactose and the like. Small amounts of other flow control agents, for example silica, may also be used. Mixtures comprising one or more of the foregoing may also be used. A number of suitable liquid carriers for dispersal of the DFM composition are available, but aqueous liquid carriers are preferred. The relative amount of dry or liquid carrier and DFM composition may vary widely, depending on flow characteristics, storage stability, cost, and the like considerations. In general the carrier will comprise about 1 to about 99 weight percent (wt %) and the DFM will comprise about 99 to about 1 wt. % of the total weight of the dispersed composition. Preferably the carrier will comprise about 10 to about 90 weight percent (wt %) and the DFM will comprise about 90 to about 10 wt. % of the total weight of the dispersed composition. Yet more preferably, the carrier will comprise about 20 to about 80 weight percent (wt %) and the DFM will comprise about 80 to about 20 wt. % of the total weight of the dispersed composition

Dispersal of the DFM in the dry or liquid carrier may be carried out by dry mixing, for example, or other known methods, for example a gravimetric or volumetric measuring device such as that disclosed in U.S. Pat. No. 4,733,971 to Pratt, where the DFM composition is controllably dispensed, metered or weighed, and subsequently delivered at predetermined rates or weights to and mixed homogeneously with a liquid aqueous carrier into which other dry particulate and liquid additive concentrates may also be delivered in predetermined quantities and concentrations. The dispersed DFM composition may be used immediately or stored prior to use. If stored, the dispersed composition may be refrigerated, isolated from the atmosphere, or the like, to preserve the viability of the microbes.

The dispersed DFM composition may optionally be diluted prior to application to the animal feed, which may itself be solid, semi-solid, or liquid. The method is of particular utility with solid or semi-solid feeds. Application may be by conventional methods, for example by draining from the mixing or storage vessel using a pump, into a feed truck that has been prefilled with a preferably known mass or volume of animal feed. As the dispersed DFM composition is added to the feed in the truck, the feed is tumbled or otherwise agitated to ensure uniform distribution of suspension throughout the feed. Afterward, the feed is delivered for presentation to the animals.

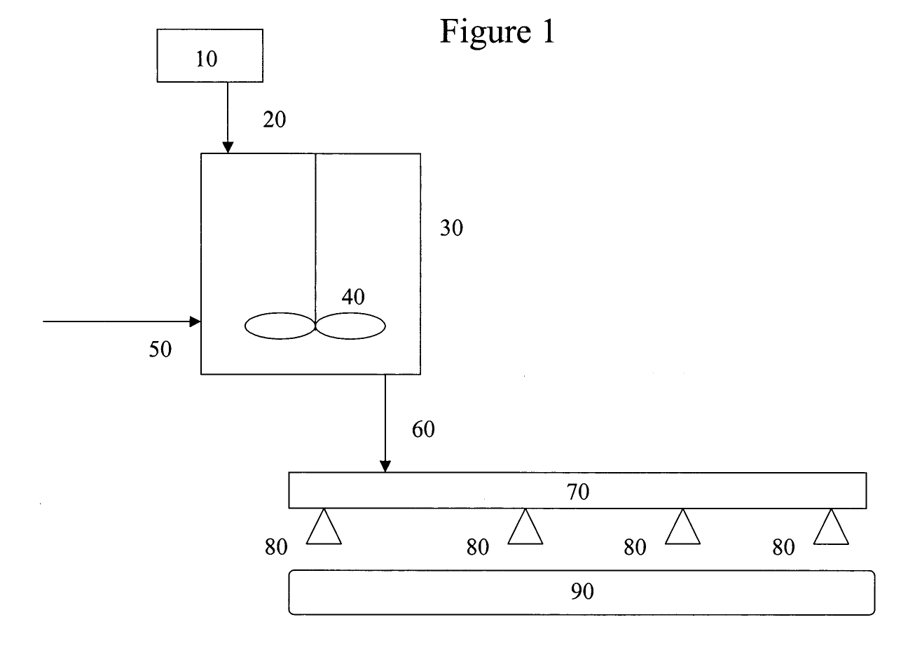

A suitable apparatus for carrying out the present method may include a mixer to prepare (and optionally store) the dispersed DFM composition and an applicator to apply the liquid to the feed source. Exemplary suitable mixers comprise a mixing instrument, such as a paddle or propeller, used in conjunction with a vessel to receive and contain the dispersed DFM composition prior to application to the feed. Exemplary suitable applicators comprise drop feeders and the like. Referring now to FIG. 1, a generalized apparatus (5) for dispersing DFM in a liquid carrier is shown. Apparatus (5) has a weighing means (10) to weigh the different components of the DFM composition. A dispensing means (20) provides communication between the storage means (not shown) and the mixing vessel (30) via the weighing means (10). The storage means stores the DFM composition and other optional components prior to weighing. The mixing vessel is equipped with a mixing means (40) and a liquid feed line (50) that introduces a liquid carrier used to form the liquid dispersed DFM. A means of delivering (60) the liquid dispersed DFM provides communication between the mixing vessel and the application means (70). The application means may comprise a plurality of nozzles (80) to provide the delivery of the liquid DFM to a vessel (90), which may be a feed vessel or feed storage vessel. It should be understood that modifications and additional components are within the scope of the present methods of administering microbial feed additives.

Multiple dry and liquid storage means for storing the various components of the DFM composition separately are especially preferred. The dry and liquid storage means include, for example, containers, tanks, vessels, hoppers, and the like, which optionally may be temperature controlled, insulated, and/or atmosphere controlled to maintain the viability of the microbes. A plurality of separate dispensing means, such as conveyor screws for the dry components and pumps for the liquid components, dispense separately and without intermingling the components from each of the storage means into a receiving means such as separate compartments of a hopper. For dry components, the dispensing means may comprise a hopper or similar device comprising vibratory means and/or agitator means to provide effective transfer of the dry component from the dispensing means. Suitable vibratory means includes devices that vibrate the dispensing means to allow flow of the dry component. Preferably the vibratory means do not interfere with the weighing means, if used. Suitable agitator means include movable paddles, fans, augers, and the like. Weighing means may be provided for determining the weights of the different components dispensed and for stopping the dispensing of each additive when a predetermined weight of that additive has been dispensed. The weigh means, for example, may comprise a weight scale means supporting the weigh hopper or supporting the storage means. The weight scale means may include a gravimetric scale.

In the apparatus described above, the weigh hopper may be scale-mounted, and the components are dispensed and weighed sequentially and cumulatively as they are added to the weigh hopper. Isolating means isolate the weighing means from movements affecting its weighing function so that accurate weight determinations are obtained. When all selected components have been dispensed into the weigh hopper and weighed, the hopper deposits its contents into another portion of the receiving means comprising a mixing vessel. All components, including the DFM, carrier, and optional additives are intermixed in the mixing vessel.

The dispersed DFM composition is then delivered to a receiving station where it is either applied directly to and mixed with a feed, or held for subsequent addition to a feed. A single application means or a plurality of application means may be used. The application means may be a drop feeder, pump, or similar device used in conjunction with known plumbing equipment, for example nozzles, to provide a consistent delivery of liquid. The pumps preferably meter liquid at a rate effective to provide appropriate amounts of the liquid DFM composition. Suitable pumps include variable-speed or displacement rotary or piston pumps.

One or more of the preceding components of the apparatus is refrigerated to maintain the DFM composition, and/or the dispersed DFM composition at a constant temperature. Exemplary components include mixing means, storage means, plumbing, and the like. When the DFM composition is prepared with a liquid carrier, a refrigeration means cools the DFM composition within a desired range, and maintains the temperature. The liquid DFM composition may be agitated to maintain a uniform dispersion of DFM in the liquid carrier to ensure a uniform concentration and to help maintain a uniform temperature.

One or more of the preceding operations, for example volumetric or gravimetric dispensing of the DFM composition to the carrier, and/or dispersed DFM composition to the feed, may be controlled by a central processing unit at the same or a remote site. The central processing unit may also be electronically interfaced with a separate weighing system.

A suitable apparatus for liquid dispersal is one available from MICRO Beef Technologies such as the META-BAC® line. Such apparatuses are described, for example, in U.S. Pat. Nos. 4,733,971; 4,815,042; 4,889,433; 4,910,024; 5,219,224; 5,340,211; 5,369,032; and 5,401,501, all of which are incorporated herein by reference. In one embodiment, water is added continuously to the dispersal machine. In another embodiment, water is added batch-wise, and the mixing/storage tank emptied completely prior to making the next batch.

In one embodiment, the method of formulating direct-fed microbials with animal feed includes dispersing a direct-fed microbial composition into a liquid carrier, storing the liquid direct-fed microbial composition at a controlled temperature, optionally with agitation; further diluting the liquid direct-fed microbial composition with an aqueous diluent; and applying at least a portion of the diluted liquid direct-fed microbial composition to animal feed. Preferably the direct-fed microbial composition is measured out prior to dispersing in a liquid carrier. Methods of measuring the composition are as described herein. The temperature the direct-fed microbial is stored at can be determined by one of ordinary skill in the art. Suitable temperatures include those that allow the direct-fed microbial to remain viable, for example about 30° F. (−1.1° C.) to about 55° F. (12.8° C.), preferably about 35° F. (1.7° C.) to about 50° F. (10° C.), and still more preferably at about 40° F. (4.4° C.) to about 45° F. (7.2° C.).

In yet another embodiment, a method of formulating direct-fed microbials with animal feed includes preparing a concentrated suspension of a known concentration and storing the suspension for extended periods of time in ready to use condition without substantial loss of microbial viability. The concentrate is preferably stored at temperatures that allow for the direct-fed microbial to remain viable. When needed, the concentrate may be administered in measured amounts, diluted with an appropriate liquid carrier, and applied or blended with feed.

In still yet another embodiment, formulations of DFM with enhanced dry delivery include E. coli 271, ATCC accession number 202020; E. coli 786, ATCC accession number 202018; E. coli 797, ATCC accession number 202019; or combinations thereof and a dry organic carrier, preferably lactose or other sugars, and wheat or corn husks, although inorganic carbonate or sulfate carriers may be used. Optionally, an amount of flow control agent may be used, e.g. silicon dioxide or metal silicates.

In one embodiment, a method of formulating direct-fed microbials with animal feed comprises dispersing a direct-fed microbial composition into a dry or liquid carrier to form a dispersed direct-fed microbial composition; and applying at least a portion of the dispersed direct-fed microbial composition to animal feed, wherein the direct-fed microbial composition comprises E. coli 271, ATCC accession number 202020; E. coli 786, ATCC accession number 202018; E. coli 797, ATCC accession number 202019; or a combination thereof.

In still another embodiment, a supplemented animal feed is formulated by formulating direct-fed microbials with animal feed by dispersing a direct-fed microbial composition into a dry or liquid carrier to form a dispersed direct-fed microbial composition; and applying at least a portion of the dispersed direct-fed microbial composition to animal feed, wherein the direct-fed microbial composition comprises E. coli 271, ATCC accession number 202020; E. coli 786, ATCC accession number 202018; E. coli 797, ATCC accession number 202019; or a combination thereof.

In yet another embodiment, a method of formulating direct-fed microbials with animal feed comprises dispersing a direct-fed microbial composition into a liquid carrier to form a liquid direct-fed microbial composition; storing the liquid direct-fed microbial composition at a controlled temperature, optionally with agitation; further diluting the liquid direct-fed microbial composition with an aqueous diluent to form a diluted liquid direct-fed microbial composition; and applying at least a portion of the diluted liquid direct-fed microbial composition to animal feed, wherein the direct-fed microbial composition comprises E. coli 271, ATCC accession number 202020; E. coli 786, ATCC accession number 202018; E. coli 797, ATCC accession number 202019; or a combination thereof.

In another embodiment, a method of excluding pathogens from an animal's gut comprises presenting a therapeutically effective amount of the supplemented animal feed prepared with the direct-fed microbial composition to the animal for consumption. A therapeutically effective amount is an amount sufficient to maintain the animal's normal gut flora.

The invention is further illustrated by the following non-limiting examples.

### EXAMPLES

In addition to a DFM used as provided by the manufacturer, three Formulations of DFM compositions are studied using liquid and dry application processes. The materials and Formulations are provided in Table 1.

TABLE 1

| | Formulations | | |
|---|---|---|---|
| | (weight | | |
| | percent) | | |
| Material, Trade name, Source | 1 | 2 | 3 |
| Calcium Sulfate, “Terra Alba”, United States Gypsum Co. | 75 | — | — |
| Calcium Carbonate 98.27%, “Limestone Code-2”, | — | 75 | — |
| Mississippi Lime Co. | | | |
| Lactose, Edible Lactose, First District Assoc. | — | — | 75 |
| Silicon Dioxide, “ZEOFREE SO”, J.M. Huber Corp. | 5 | 5 | 5 |
| E. coli Culture, Lot 100002, E. coli 786, accession number | 20 | 20 | 20 |
| 202018 and E. coli, 797, accession number 202019, | | | |
| Alpharma Inc. | | | |

In Example 1, DFM (E. coli 786, accession number 202018 and E. coli, 797, accession number 202019 as supplied by the manufacturer) is dispersed in water and applied directly to feed using a META-BAC® machine from Access Micro Systems supplied by Micro-Beef Technologies. The liquid DFM composition is maintained at a constant temperature, and is pumped into the feed as the feed is mixed. The DFM is observed to go into solution well. As the META-BAC® tank emptied, no DFM remains and the tank cleans easily.

In Example 2, 200 grams of Formulation 3 is added directly to 10 pounds (4.5 kilograms) of water and mixed in a META-BAC® machine using a stainless steel paddle and a plastic float containing a mercury switch to keep a constant level of water in the tank. The water is maintained at a constant temperature of 36 to 42° F. (2.2 to 5.6° C.) using a cooling unit and a temperature-probe. The silicon dioxide and bacterial culture are easily dispersed in the water, with no formation of foam. The solution of DFM is added to feed in a bowl. As illustrated herein, the liquid application of Formulation 3 provides a convenient and simple method of dispersing a desired amount of DFM to feed uniformly as the lactose dissolved in the water while the silicon dioxide and bacterial culture dispersed easily in the water. The liquid application method also results in lower waste as substantially all of the DFM is provided to the feed. No extra DFM is required unlike “dry feed” application equipment that deliver the DFM via augers where DFM material can get trapped, as is illustrated in Comparative Examples 3 and 4 herein.

In Comparative Examples 3 and 4, the flowability of the DFM composition in a dry-feed machine is assessed by measuring the variability of the weights of product collected over specific time periods. A Dry Material Feeder manufactured by AccuRate of Whitewater, Wis. is used, fitted with an auger to meter the amount of DFM to be added per ton of feed. A carrier without DFM is used to calibrate auger speed and time so that the correct amount of dry material is delivered. Dry product is discharged into a bowl that is flushed with water to better distribute the DFM onto the feed.

Table 2 shows the results when 250 grams of DFM (E. coli culture as provided by the manufacturer, Comparative Example 3) are placed in the feeder and the amount of dry DFM processed through the auger at 10-second intervals is weighed.

TABLE 2

| | | High speed | High speed |
|---|---|---|---|
| Interval | Low speed | delivery of | delivery of new |
| No. | delivery (g) | same material (g) | material** (g) |
| 1 | .72 | 2.53 | 2.77 |
| 2 | .69 | 2.52 | 2.79 |
| 3 | .67 | 2.525 | 2.84 |
| 4 | .70 | 2.54* | 2.76 |
| 5 | .67 | 2.57 | 2.86 |
| 6 | .67 | 2.57 | 2.82 |
| 7 | .67 | 2.56 | 2.87 |
| 8 | .69 | 2.56 | 2.86 |
| 9 | .67 | 2.57 | 2.89 |
| 10 | .675 | 2.575 | 2.87 |

*A small amount of E. coli culture that built up on machine fell off.
**Equipment was cleaned (wiped out) before new material was added.

Because the auger must be allowed to fill before the equipment can deliver the correct amount of dry DFM, and the feeder bin must be maintained at a level where the DFM continues to fill the auger, there must be more DFM used in the delivery than is needed to dose the animals, which results in waste. As the number of intervals increases, the DFM begins to stick to and build up on the auger equipment. It is observed that the DFM also shows signs of separation in the feeder bin, and does not look consistent, although the equipment cleans well.

Table 3 shows the results when 250 grams of DFM (E. coli culture as provided by the manufacturer, Comparative Example 4) is placed in the feeder and the amount of dry DFM processed through the auger at 30-second intervals is weighed.

TABLE 3

| | Low speed | High speed delivery |
|---|---|---|
| Interval No. | delivery (g) | new material* (g) |
| 1 | 4.5 | 13.9 |
| 2 | 4.4 | — |
| 3 | 4.2 | 13.9 |
| 4 | 4.2 | 13.9 |
| 5 | 4.1 | 13.8 |
| 6 | 4.1 | 13.9 |
| 7 | 4.0 | 13.9 |
| 8 | 4.0 | 13.8 |
| 9 | — | 14.0 |
| 10 | — | 13.7 |
| 11 | — | 13.7 |
| 12 | — | 13.9 |

*Auger is changed before new material was added.

The equipment used for Comparative Example 4 does not clean up well. When water is used for cleaning, the DFM becomes sticky, which makes it more difficult to work with.

Examples 5 and 6 illustrate flowability of dry, dispersed DFM compositions using a Dry Material Feeder manufactured by AccuRate of Whitewater, Wis. Each Example is run at one of two different facilities using the same type of equipment.

In Example 5, Formulations 1-3 are separately dispensed in 200 to 500 gram amounts into a 7-inch (17.78 centimeter) square stainless steel hopper with a r liner of an AccuRate Dry Material Feeder. Each Formulation is fed from the hopper by a ¾-inch (1.9 cm) stainless steel auger with an open-flight screw thread along an 8-inch (20.32) stainless steel tube. One side of the rubber liner is connected to a steel paddle that is connected to the rotating auger, such that the rotation of the auger causes the paddle to rotate. This moves the rubber liner and ensures that the product is agitated and moved to the bottom of the hopper. By this means, each Formulation is fed into a tared-weight plastic container which is then weighed on an OHAUS Champ II top-loading balance (Model number CD11) over a specified time period. A DC Motor Tester supplied by Lextron from Allied Electronics is used for measuring the voltage and by this means the speed of the auger may be varied. The speed is varied to give a delivery of product in 10 to 20 seconds. The delivery time (in seconds) is set and measured using an Eagle Signal Timer (Code Number CX202A6-27861). The capacity of the machine is approximately 3 kg.

The variance in weights is measured over a number of runs at both slow and fast speeds (10 or 24 volts) to arrive at a calculation for the Percent Coefficient of Variation (% CV):

$$\% CV = \frac{Standard Deviation for the weight in grams}{average weight in grams} \times 100$$

This calculation is then repeated with reused product at high speed (24 volts) to see if reusing the product through the machine altered any of the flow characteristics.

Table 4 provides the results of the flowability study for Formulation 1 (Example 5). After formulation, the DFM composition was stored at about 0° C. for about seven days until it was used.

TABLE 4

| | 10 Volts | 24 Volts | 24 Volts |
|---|---|---|---|
| | Low Speed | High Speed | High Speed* |
| Run No. | 20 seconds | 20 seconds | 20 seconds |
| 1 | 0.025 | 0.052 | 0.054 |
| 2 | 0.023 | 0.064 | 0.053 |
| 3 | 0.023 | 0.056 | 0.063 |
| 4 | 0.024 | 0.06 | 0.06 |
| 5 | 0.022 | 0.062 | 0.063^{a} |
| 6 | 0.019^{c} | 0.058 | 0.044 |
| 7 | 0.019^{c} | 0.048 | 0.056^{d} |
| 8 | 0.016^{c} | 0.061 | 0.064 |
| Mean | 0.021375 | 0.057625 | 0.057125 |
| St. Dev. | 0.003068 | 0.005397 | 0.006813 |
| C.V. | 14.35176 | 9.365307 | 11.92567 |

*Reused material
^{a}added additional 100 grams of reused product
^{b}added additional 184 grams of reused product
^{c}The weights dropped, approximately 10% of hopper was filled, another 168 grams was added to the hopper & ran batches 9, 10 & 11 with results of #9 low speed (LS) 0.016, #10 LS 0.017 & #11 LS 0017; the voltage was at 10 Volts; no reason noted for this delivery drop

Table 5 provides the results of the flowability study for Formulation 2 (Example 5). The product appears stickier, but the machine agitation makes flow acceptable.

TABLE 5

| | 10 Volts | 24 Volts | 24 Volts |
|---|---|---|---|
| | 20 Seconds | 20 seconds | 20 seconds |
| Run No. | Low Speed | High Speed | High Speed* |
| 1 | 0.026 | 0.06 | 0.057^{c} |
| 2 | 0.034 | 0.073 | 0.076 |
| 3 | 0.033 | 0.071 | 0.078^{d} |
| 4 | 0.031 | 0.074 | 0.088^{e} |
| 5 | 0.025 | 0.065^{a} | 0.06^{f} |
| 6 | 0.024^{b} | 0.074 | 0.079 |
| 7 | 0.025 | 0.076 | 0.087 |
| 8 | 0.024 | 0.079 | 0.093 |
| Mean | 0.02775 | 0.0715 | 0.07725 |
| St. Dev. | 0.0042 | 0.006164 | 0.012937 |
| C.V. | 15.13636 | 8.621558 | 16.74648 |

*Reused
^{a}added 2 of 2 approximately 500 grams to hopper
^{b}dribble noted - could affect weight reused high speed (RHS) 750 grams of material was used for initial hopper charge
^{c}weight appears low - no cause noted
^{d}convex at delivery end of auger
^{e}concave at delivery end of auger
^{f}convex at delivery end of auger
Product 1 & 2 both appeared to surge out of delivery auger vs. a smooth flow

Table 6 provides the results of the flowability study for Formulation 3 (Example 5).

TABLE 6

| | 10 Volts | 24 volts | 24 volts |
|---|---|---|---|
| | 20 seconds | 20 seconds | 20 seconds |
| Run No. | Low Speed | High Speed | High Speed* |
| 1 | 0.031 | 0.094 | 0.095 |
| 2 | 0.033 | 0.096 | 0.096 |
| 3 | 0.031 | 0.095 | 0.094 |
| 4 | 0.033 | 0.095 | 0.096 |
| 5 | 0.03^{b} | 0.095^{a} | 0.096 |
| 6 | | 0.095 | 0.097 |
| 7 | | 0.095 | 0.097^{c} |
| 8 | | 0.097 | 0.096^{d} |
| Mean | 0.0316 | 0.09525 | 0.095875 |
| St. Dev. | 0.001342 | 0.000886 | 0.000991 |
| C.V. | 4.245699 | 0.930609 | 1.03367 |

^{a}added 2 of 2 additional 500 grams
^{b}auger was slightly showing at this delivery ran out of product to complete run due to flow
^{c}added 200 grams of RHS to hopper
^{d}added 100 grams of RHS to hopper

As may be seen from the results for Example 5, the lactose-based Formulation (Formulation 3) produces the lowest CV values (4.2% at low speed and 0.9% at high speed). Furthermore, it is noted that the product flowed evenly from the delivery tube vs. the surge effect noted with Formulations 1 and 2. The calcium sulfate and carbonate Formulations (Formulations 1 and 2) produced high CV values (14.4 and 15.1% for slow speed and 9.4 and 8.6% for high speed, respectively). The lactose-based Formulation 3 was thus more reproducible between flow studies compared to the inorganic Formulations 1 and 2 and also flowed at a faster rate.

The procedures used in Example 5 are also used for Example 6, including the same type of equipment. The Formulations are fed into a tared-weight plastic container which is then weighed on an AND Model top-loading balance (Model number SV610) over a specified time period. A DC Motor Tester and Timer, supplied by Nutrition Physiology, is used for measuring and adjusting the voltage and by this means adjusting the speed of the auger so as to deliver product in 18 to 27 seconds. The variance in weights is measured over a number of runs to arrive at the Percent Coefficient of Variation (% CV) as described above.

Table 7 provides the results of the flowability study for Formulation 1 (Example 6). It is started with a ½-inch auger at 20 seconds, which delivered 1 gram, then switched to ¾-inch Auger, whereupon the premix surges out of the end of the auger at high speed. The 1st 20 seconds=36 grams, next 20 seconds a rat hole in hopper with 33.9 grams delivery, next 20 seconds delivers 33.2 grams.

TABLE 7

| | 27 seconds | 18 seconds | 18 seconds |
|---|---|---|---|
| Run No. | Low Speed | High Speed | High Speed* |
| 1 | 9.3 | 30.7 | 30.1^{c} |
| 2 | 9.1 | 30.6 | 34 |
| 3 | 9.4 | 29.5 | 33.6 |
| 4 | 9.1 | 29.9 | 32.5 |
| 5 | 9.8 | 30.8 | 33.7 |
| 6 | 8.8^{a} | 30.4 | 32 |
| 7 | 9.7 | 32.8 | 34.8 |
| 8 | ^{b} | 32.5 | 35.2 |
| Mean | 9.314266 | 30.9 | 33.2375 |
| St. Dev. | 0.353217 | 1.16619 | 1.653514 |
| C.V. | 3.792202 | 3.774079 | 4.974843 |

*Reused
^{a}getting very low in hopper
^{b}ran product out of hopper (67.9 grams left - too low for accuracy); small amount of compaction around end of auger when empty.
^{c}auger probably not completely loaded

Table 8 provides the results of the flowability study for Formulation 2 (Example 6).

TABLE 8

| | 27 sec | 18 sec | 18 sec |
|---|---|---|---|
| Run No. | Low Speed | High Speed | High Speed* |
| 1 | 12.8 | 28.9 | 26.8 |
| 2 | 13.6 | 30.9 | 25.1 |
| 3 | 13.1 | 35.3 | 20.9 |
| 4 | 12.8^{a} | 34.2 | 28.5 |
| 5 | | 36.8 | 28.5 |
| 6 | | 38 | 29.1 |
| 7 | | 37.4 | 28.7 |
| 8 | | 38.4 | 28.1 |
| Mean | 13.075 | 34.9875 | 26.9625 |
| St. Dev. | 0.377492 | 3.470462 | 2.773825 |
| C.V. | 2.887126 | 9.919149 | 10.28772 |

*Reused
^{a}holding on auger, therefore light weight

The CV values for Formulations 1 and 2 of Example 6 were somewhat improved with values varying from 3.8 and 2.9% for slow speeds and 2.9 and 9.9% for fast speeds, respectively.

In Example 7, water uptake by Formulations 1-3 under high humidity is determined by recording the weight of a small, open-top container of approximately 100 grams capacity, filling with approximately 100 grams of the test Formulation, and calculating the weight of the test material. The small container is then placed in a larger container and water was added to the large container in an amount sufficient to cover the bottom of the large container without floating the small container. A plastic cover is then placed over the large container for the duration of the test. The start date and time are recorded and at the end of the test period (4 days) the small container is removed, the outside carefully dried, and the gross weight determined. The net weight and percent change in weight is then calculated and the test materials are examined visually to determine any change in physical characteristics. Data from the humidity study is shown in Table 9.

TABLE 9

| | Net Wt. of | Change in | % Change |
|---|---|---|---|
| | Sample (g) | Net Wt. (g) | in Wt. |
| Formulation 1 | | | |
| Start of Test | 99.0 | | |
| End of Test | 105.6 | 6.60 | 6.67 |
| Formulation 2 | | | |
| Start of Test | 98.4 | | |
| End of Test | 104.2 | 5.80 | 5.89 |
| Formulation 3 | | | |
| Start of Test | 99.2 | | |
| End of Test | 105.0 | 5.80 | 5.85 |

After 4 days at room temperature and high humidity conditions, all three Formulations showed no visible change in physical characteristics. Formulation 3 (lactose) showed a very small amount of compaction and was not as easily removed from the container compared to Formulations 1 and 2. All Formulations absorbed approximately the same amounts of water; the variation was only from 5.85 to 6.67%.

Bacterial viability and homogeneity studies are run for the dry dispersed compostions by taking samples in small plastic sterile WhirlpaK bags over various time periods of the dry feed testing. These samples are submitted for plate counts of the E. coli using MacConkey Sorbitol agar. Results are shown in the following tables.

E. coli bacterial
counts (colony
forming units
per gram)

| | | |
|---|---|---|
| Formulation:Sample:Speed | 10⁹ Plate | 10¹⁰ Plate |

Example 5

| | | |
|---|---|---|
| 2 | 179 | 9 |
| 2:2:LS | 155 | 13 |
| 2:4:LS | 217 | 33 |
| 2:6:LS | 203 | 22 |
| 2:8:LS | 139 | 15 |
| Avg. (cfu/g) | 179 | 21 |
| St. Dev. | 37 | 9 |
| % RSD | 21 | 45 |
| 2:2:HS | 191 | 14 |
| 2:4:HS | 184 | 40 |
| 2:6:HS | 151 | 9 |
| 2:8:HS | 166 | 12 |
| Avg. (cfu/g) | 173 | 19 |
| St. Dev. | 18 | 14 |
| % RSD | 10 | 76 |
| 2:2:RHS | 155 | 14 |
| 2:4:RHS | 136 | 8 |
| 2:6:RHS | 162 | 15 |
| 2:8:RHS | 132 | 23 |
| Avg. (cfu/g) | 146 | 15 |
| St. Dev. | 15 | 6 |
| % RSD | 10 | 41 |
| Grand avg. (cfu/g) | 166 | 18 |
| St. Dev. (Total) | 26 | 10 |
| % RSD (Total) | 16 | 54 |
| 3 | 190 | 16 |
| 3:2:LS | 133 | 22 |
| 3:4:LS | 132 | 13 |
| 3:6:LS | | |
| 3:8:LS | | |
| Avg. (cfu/g) | 133 | 18 |
| St. Dev. | 1 | 6 |
| % RSD | 1 | 36 |
| 3:2:HS | 185 | 21 |
| 3:4:HS | 145 | 15 |
| 3:6:HS | 178 | 18 |
| 3:8:HS | 189 | 15 |
| Avg. (cfu/g) | 174 | 17 |
| St. Dev. | 20 | 3 |
| % RSD | 11 | 17 |
| 3:2:RHS | 155 | 18 |
| 3:4:RHS | 105 | 6 |
| 3:6:RHS | 173 | 8 |
| 3:8:RHS | 162 | 15 |
| Avg. (cfu/g) | 149 | 12 |
| St. Dev. | 30 | 6 |
| % RSD | 20 | 48 |
| Grand avg. (cfu/g) | 156 | 15 |
| St. Dev. (Total) | 27 | 5 |
| % RSD (Total) | 17 | 34 |

Example 6

| | | |
|---|---|---|
| 1 | 153 | 22 |
| 1:2:LS | 223 | 41 |
| 1:4:LS | 211 | 27 |
| 1:6:LS | 133 | 27 |
| 1:8:LS | | |
| Avg. (cfu/g) | 189 | 32 |
| St. Dev. | 49 | 8 |
| % RSD | 26 | 26 |
| 1:2:HS | 154 | 28 |
| 1:4:HS | 148 | 13 |
| 1:6:HS | 138 | 21 |
| 1:8:HS | 142 | 14 |
| Avg. (cfu/g) | 146 | 19 |
| St. Dev. | 7 | 7 |
| % RSD | 5 | 37 |
| 1:2:RHS | TNTC | 127 |
| 1:4:RHS | 171 | 17 |
| 1:6:RHS | 150 | 12 |
| 1:8:RHS | 162 | 73 |
| Avg. (cfu/g) | 161 | 57 |
| St. Dev. | 11 | 54 |
| % RSD | 7 | 94 |
| 2 | 767 | 21 |
| 2:2:LS | 114 | 19 |
| 2:4:LS | 187 | 24 |
| 2:6:LS | | |
| 2:8:LS | | |
| Avg. (cfu/g) | 151 | 22 |
| St. Dev. | 52 | 3 |
| % RSD | 34 | 12 |
| 2:2:HS | 159 | 13 |
| 2:4:HS | 564 | 63 |
| 2:6:HS | 145 | 8 |
| 2:8:HS | 165 | 19 |
| Avg. (cfu/g) | 258 | 26 |
| St. Dev. | 204 | 25 |
| % RSD | 79 | 98 |
| 2:2:RHS | 177 | 12 |
| 2:4:RHS | 383 | 11 |
| 2:6:RHS | 557 | 41 |
| 2:8:RHS | 152 | 47 |
| Avg. (cfu/g) | 317 | 28 |
| St. Dev. | 190 | 19 |
| % RSD | 60 | 68 |

Overall, the bacteria counts are consistent and survival rates in the formulations are good.

In Example 8 the application of the DFM formulations to cattle feed at a feed mill is observed. The mill can accommodate 72,000 to 77,000 cattle at one time and is capable of processing 1.7 million pounds of cattle feed per day. The treated and mixed cattle feed is discharged into feed trucks and then delivered to concrete troughs where the cattle have access from a number of pens. The feed consists of primarily of a corn and alfalfa mix. The corn is treated with steam to break open the kernels thus making it more digestible for the cattle. The treated feed, which had a measured temperature of around 121° F. (49° C.) after steam treatment in the mixer, is transferred to a finished feed bin. The temperature of the feed in transit to the finished feed bin is reduced to approximately 96° F. (36° C.) within a few minutes. The E. coli bacteria have a growth-range up to 130° F. (54° C.), suggesting that the holding time of the feed at the higher temperature is preferably only minutes. Preferably, the application of the dispersed bacterial culture is as a water spray of the liquid dispersion as the feed is mixed in the plant and sent to the finished feed bin. The feed delivery trucks are loaded typically within 15 minutes to 4 hours for delivery of feed to cattle. The next days early morning feeding is manufactured in the early afternoon of the previous day. In this instance manufactured feed may be held in a finished feed bin for up to 12 to 14 hours at this lower temperature of 96° F. (36° C.) or less.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of formulating direct-fed microbials with animal feed comprising
dispersing a direct-fed microbial composition into a dry or liquid carrier to form a dispersed direct-fed microbial composition; and
applying at least a portion of the dispersed direct-fed microbial composition to animal feed,
wherein the direct-fed microbial composition comprises E. coli 271, ATCC accession number 202020; E. coli 786, ATCC accession number 202018; E. coli 797, ATCC accession number 202019; or a combination thereof.

2. The method of claim 1, wherein the liquid carrier comprises water.

3. The method of claim 1, wherein the animal feed is livestock feed.

4. The method of claim 1, wherein a known amount of the direct-fed microbial composition is dispersed into a known amount of carrier.

5. The method of claim 1, wherein the dispersed direct-fed microbial composition is applied to a known amount of animal feed.

6. The method of claim 1, further comprising further diluting the dispersed direct-fed microbial composition prior to applying.

7. The method of claim 1, further comprising storing the dispersed direct-fed microbial composition prior to applying.

8. A supplemented animal feed formulated by the method of claim 1.

9. A method for administering a direct-fed microbial composition to a population of animals, comprising presenting the supplemented animal feed of claim 8 to the population of animals for consumption.

10. A method of formulating direct-fed microbials with animal feed, comprising
dispersing a direct-fed microbial composition into a liquid carrier to form a liquid direct-fed microbial composition;
storing the liquid direct-fed microbial composition at a controlled temperature, optionally with agitation;
further diluting the liquid direct-fed microbial composition with an aqueous diluent to form a diluted liquid direct-fed microbial composition; and
applying at least a portion of the diluted liquid direct-fed microbial composition to animal feed,
wherein the direct-fed microbial composition comprises E. coli 271, ATCC accession number 202020; E. coli 786, ATCC accession number 202018; E. coli 797, ATCC accession number 202019; or a combination thereof.

11. The method of claim 10, wherein a known amount of the direct-fed microbial composition is dispersed into a known amount of liquid carrier.

12. The method of claim 10, further comprising measuring an amount of direct-fed microbial composition prior to dispersing to form a known amount of direct-fed microbial composition.

13. The method of claim 11, wherein the measuring, dispersing, and storing are performed with a single apparatus.

14. The method of claim 10, wherein dispersing, storing, diluting, optional agitation, and applying are performed with a single apparatus.

15. The method of claim 10, wherein the temperature at least one of dispersing, storing, diluting, optional agitation, and/or applying is controlled in the range of 30-45° F.

16. The method of claim 10, wherein at least one of dispersing, storing, diluting, optional agitation, and/or applying is controlled with a microprocessor.

17. The method of claim 10, wherein the controlled temperature is in the range of 30-45° F.

18. The method of claim 10, wherein applying at least a portion of the diluted liquid direct-fed microbial composition to animal feed is a known amount of direct-fed microbial composition.

19. A method of excluding pathogens from an animal's gut, comprising presenting a therapeutically effective amount of the supplemented animal feed of claim 8 to the animal for consumption.
